(19) United States
(12) Patent Application Publication
(10) Pub. No.: US 2026/0016491 A1
(43) Pub. Date: Jan. 15, 2026

(54) METHODS FOR THE TREATMENT OF SYMPTOMS OF NEUROLOGICAL AND MENTAL HEALTH DISORDERS

(71) Applicant: Curemark, LLC, Rye Brook, NY (US)

(72) Inventor: Joan M. Fallon, White Plains, NY (US)

(21) Appl. No.: 19/335,455

(22) Filed: Sep. 22, 2025

Related U.S. Application Data
(63) Continuation of application No. 17/235,646, filed on Apr. 20, 2021, now abandoned.
Application No. 17/235,646 is a continuation of application No. 13/002,136, filed on Apr. 13, 2011, now Pat. No. 11,016,104.
(60) Provisional application No. 61/077,463, filed on Jul. 1, 2008.

(51) Int. Cl. G01N 33/68; A61K 38/46; A61K 38/47; A61K 38/48; A61K 38/50

(52) U.S. Cl. CPC G01N 33/6896; A61K 38/465; A61K 38/47; A61K 38/48; A61K 38/4826; A61K 38/4873; A61K 38/488; A61K 38/50; G01N 2333/976; G01N 2800/2821; G01N 2800/304

(57) ABSTRACT

A therapeutic composition for the treatment of the symptoms of neurological and mental health disorders, such as Alzheimer's disease, bipolar disorder, obsessive compulsive disorder, and oppositional defiant disorder, and the method for preparing the therapeutic agents is disclosed. The therapeutic agent is a stable pharmaceutical preparation containing, but not limited to, digestive/pancreatic enzymes. The therapeutic agent may be manufactured by a variety of encapsulation technologies. Delivery of the therapeutic agent may be made orally, through injection, by adherence of a medicated patch or other method. Further, a method of using fecal chymotrypsin level as an indicator of the presence of neurological and mental health disorders, such as Alzheimer's disease, bipolar disorder, obsessive compulsive disorder, and oppositional defiant disorder, or the likelihood of an individual to develop these disorders is disclosed.

### CROSS-REFERENCE TO RELATED APPLICATIONS

This application is a continuation of U.S. patent application Ser. No. 17/235,646, filed Apr. 20, 2021, which is a continuation of U.S. patent application Ser. No. 13/002,136, filed Apr. 13, 2011, now U.S. Pat. No. 11,016,104, which is a § 371 national stage entry of International Application No. PCT/US2009/049374, filed Jul. 1, 2009, which claims the benefit of U.S. Provisional Application No. 61/077,463, filed Jul. 1, 2008, incorporated by reference herein in their entirety.

### TECHNICAL FIELD

This disclosure relates to a treatment for the symptoms of selected neurological and mental health disorders, such as Alzheimer's disease, bipolar disorder, obsessive compulsive disorder, and oppositional defiant disorder, and more particularly, to the use of a pharmaceutical composition comprising one or more digestive enzymes, such as pancreatic enzymes, in the treatment of the symptoms of these disorders. The disclosure also relates to a method of making the same. The disclosure further relates to the use of an individual's fecal chymotrypsin level as an indicator, e.g., biomarker of whether these disorders may be present in an individual or whether the individual has a propensity to develop the disorder.

### BACKGROUND

Dysautonomias can result in symptoms in which one or more areas of the body are innervated by the autonomic nervous system. While some dysautonomias are well known, other conditions have yet to be determined as a dysautonomia.

Symptoms of known dysautonomias include palpitations, chest pain, tachycardia, excessive fatigue, severe fluctuations in blood pressure, excessive sweating, fainting, exercise intolerance, shortness of breath, visual disturbances including blurred vision, tunneling, and double vision, migraines, dizziness, insomnia, gastrointestinal problems including diarrhea, and constipation, bloody stools, fainting/near fainting, frequent urination, convulsions, and cognitive impairment. Secondarily, other symptoms such as depression, dysthymia, obsessive compulsive tendencies, and difficulty with ambulation and other symptoms may also be a part of the dysautonomic picture.

Conditions such as familial dysautonomia (FD), also known also as Riley-Day syndrome, Parkinson's disease, Guillain-Barré syndrome (GBS), Dopamine-b-Hydroxylase deficiency, baroreflex failure, Guillain-Barré Syndrome, neuroblastoma and other tumors which affect the neuroendocrine system, Aromatic L-Amino Acid Decarboxylase deficiency, Tetrahydrobiopterin deficiency, Familial Paraganglioma syndrome, “Shy-Drager Syndrome,” also referred to as “Multiple System Atrophy” or MSA, Neurally Mediated Syncope, also known as Neurocardiogenic Syncope, fetal fatal insomnia (FFI), diabetic cardiovascular neuropathy, hereditary sensory and autonomic neuropathy type III (HSAN III), Menke's disease, monoamine oxidase deficiency states, and other disorders of dopamine metabolism, dysautonomic syndromes and disorders of the cardiovascular system, Chagas disease, diabetic autonomic failure, and pure autonomic failure, are well known as conditions associated with or primarily due to a dysautonomia.

Alzheimer's disease (AD) is a progressive, neurodegenerative disease characterized in the brain by abnormal clumps (amyloid plaques) and tangled bundles of fibers (neurofibrillary tangles) composed of misplaced proteins. Age is the most important risk factor for AD; the number of people with the disease doubles every 5 years beyond age 65. Three genes have been discovered that cause early onset (familial) AD. Other genetic mutations that cause excessive accumulation of amyloid protein are associated with age-related (sporadic) AD. Symptoms of AD include memory loss, language deterioration, impaired ability to mentally manipulate visual information, poor judgment, confusion, restlessness, and mood swings. Eventually AD destroys cognition, personality, and the ability to function. The early symptoms of AD, which include forgetfulness and loss of concentration, are often missed because they resemble natural signs of aging.

There is no cure for AD and no way to slow the progression of the disease. For some people in the early or middle stages of AD, medication such as tacrine (COGNEX®) may alleviate some cognitive symptoms. Donepezil (ARICEPT®), rivastigmine (EXELON®), and galantamine (Reminyl) may keep some symptoms from becoming worse for a limited time. A fifth drug, memantine (NAMENDA®), was recently approved for use in the United States. Combining memantine with other AD drugs may be more effective than any single therapy. One controlled clinical trial found that patients receiving donepezil plus memantine had better cognition and other functions than patients receiving donepezil alone. Also, other medications may help control behavioral symptoms such as sleeplessness, agitation, wandering, anxiety, and depression.

Bipolar disorder symptoms are characterized by an alternating pattern of emotional highs (mania) and lows (depression). The intensity of signs and symptoms can vary from mild to severe. There may even be periods when the individual does not seem affected at all. Bipolar disorder symptoms reflect a range of moods from severe mania to hypomania, to balanced to mild/moderate depression, to severe depression.

Signs and symptoms of the manic phase of bipolar disorder may include euphoria, extreme optimism, inflated self-esteem, poor judgment, rapid speech, racing thoughts, aggressive behavior, agitation, increased physical activity, risky behavior, spending sprees, increased drive to perform or achieve goals, increased sexual drive, decreased need for sleep, tendency to be easily distracted, inability to concentrate, and drug abuse.

Signs and symptoms of the depressive phase of bipolar disorder may include sadness, hopelessness, suicidal thoughts or behavior, anxiety, guilt, sleep problems, appetite problems, fatigue, loss of interest in daily activities, problems concentrating, irritability, and chronic pain without a known cause.

Bipolar disorder is divided into two main subtypes, Bipolar I and Bipolar II.

Bipolar I disorder: An individual has experienced at least one manic episode, with or without previous episodes of depression.

Bipolar II disorder: An individual has experienced at least one episode of depression and at least one hypomanic episode. A hypomanic episode is similar to a manic episode but much briefer, lasting only a few days, and not as severe. With hypomania, the individual may have an elevated mood, irritability and some changes in the individual's functioning, but generally can carry on with his normal daily routine and functioning and does not require hospitalization. In bipolar II disorder, the periods of depression are typically much longer than the periods of hypomania.

Cyclothymia is a mild form of bipolar disorder. Cyclothymia includes mood swings, but the highs and lows are not as severe as those of full-blown bipolar disorder.

Some people with bipolar disorder have rapid cycling bipolar disorder. This is the occurrence of four or more mood swings within 12 months. These moods shifts can occur rapidly, sometimes within just hours. In mixed state bipolar disorder, symptoms of both mania and depression occur at the same time.

Severe episodes of either mania or depression may result in psychosis, or a detachment from reality. Symptoms of psychosis may include hearing or seeing things that are not there (hallucinations) and false but strongly held beliefs (delusions).

It is not known what causes bipolar disorder. But a variety of biochemical, genetic and environmental factors seem to be involved in causing and triggering bipolar episodes:

Biochemical. Some evidence from high-tech imaging studies indicates that people with bipolar disorder have physical changes in their brains. The significance of these changes is still uncertain but may eventually help pinpoint causes. The naturally occurring brain chemicals called neurotransmitters, which are tied to mood, also may play a role. Hormonal imbalances also are thought to be a culprit.

Genes. Some studies show that bipolar disorder is more common in people whose biological family members also have the condition. Researchers are trying to find genes that may be involved in causing bipolar disorder. Some studies also show links between bipolar disorder and schizophrenia, pointing to a shared genetic cause.

Environment. Environment also is thought to play a causal role in some way. Some studies of identical twins show that one twin has the condition while the other does not—which means genes alone are not responsible for bipolar disorder. Environmental causes may include problems with self-esteem, significant loss or high stress.

It is estimated that about 1 percent of the population has bipolar disorder. However, some researchers suggest that bipolar disorder occurs on a continuum, and that many more people may have other forms of the disorder, pushing its prevalence as high as 6 percent of the population. In addition, some people may go undiagnosed because they do not seek treatment, because their condition is mistaken for depression or because their symptoms do not meet current diagnostic criteria.

Bipolar I disorder affects about the same number of men and women, but bipolar II, the rapid cycling form, is more common in women. In either case, bipolar disorder usually starts between ages 15 and 30.

Factors that may increase the risk of developing bipolar disorder include having other biological family members with bipolar disorder, periods of high stress, drug abuse, and major life changes, such as the death of a loved one.

Medications are a vital part of bipolar treatment. Because medications for bipolar disorder can cause serious but rare side effects, individuals may be reluctant to take medications.

Medication options include Mood Stabilizers, Anti-seizure Medications, Antidepressants, and other medications.

Mood Stabilizers. Mood stabilizers are most the commonly prescribed medications for bipolar disorder. These medications help regulate and stabilize mood so that the person does not swing between depression and mania. Lithium (ESKALITH®, LITHOBID®) has been widely used as a mood stabilizer and is generally the first line of treatment for manic episodes. A doctor may recommend that mood stabilizers be taken for the rest of the person's life to prevent and treat manic episodes.

Anti-seizure Medications. Anti-seizure medications are used to prevent mood swings, especially in people with rapid cycling bipolar disorder. These medications, such as valproic acid (DEPAKENE®), divalproex (DEPAKOTE®) and lamotrigine (LAMICTAL®), also are widely used as mood regulators. These medications are also known as anticonvulsants.

Antidepressants. Use of antidepressants in bipolar disorder, although once common, is now controversial. Antidepressants may not be advised at all, depending on the situation. There is limited data indicating that antidepressants are effective for bipolar disorder and, in some cases, they can trigger manic episodes.

Other Medications. Certain atypical antipsychotic medications, such as olanzapine (Zyprexa) and risperidone (Risperdal), may help people who do not gain benefits from anti-seizure medications. And anti-anxiety medications, such as benzodiazepines, may help improve sleep. In addition, one medication, quetiapine (SEROQUEL®), has been approved by the Food and Drug Administration to treat both the manic and depressive episodes of bipolar disorder.

Numerous medications are available to treat bipolar disorder. A doctor may advise combining certain medications for maximum effect.

All medications have side effects and possible health risks. Certain antipsychotic medications, for instance, may increase the risk of diabetes, obesity and high blood pressure. A doctor should monitor a patient for health problems. Also, mood-stabilizing medications may harm a developing fetus or nursing infant. Women with bipolar disorder who want to become pregnant or do become pregnant must fully explore with their health care providers their options and the benefits and risks of medications.

Obsessive-compulsive disorder (OCD) is a type of anxiety disorder. Obsessive-compulsive disorder symptoms include both obsessions and compulsions. OCD obsessions are repeated, persistent, unwanted ideas, thoughts, images or impulses that the individual experiences involuntarily and that appear to be senseless. These obsessions typically intrude when the individual is trying to think of or do other things. Typical OCD obsessions revolve around: Fear of contamination or dirt, Repeated doubts, Having things orderly and symmetrical, Aggressive or horrific impulses, and Sexual images.

OCD symptoms involving obsessions may include: Fear of being contaminated by shaking hands or by touching objects others have touched, Doubts that the individual has locked the door or turned off the stove, Repeated thoughts that the individual has hurt someone in a traffic accident, Intense distress when objects are not orderly, lined up properly or facing the right way, Images of hurting the individual's child, Impulses to shout obscenities in inappropriate situations, Avoidance of situations that can trigger obsessions, such as shaking hands, Replaying pornographic images in the individual's mind, Dermatitis because of frequent hand washing, Skin lesions because of picking at the skin, and Hair loss or bald spots because of hair pulling.

Compulsions are repetitive behaviors that the individual feels driven to perform. These repetitive behaviors are meant to prevent or reduce anxiety or distress related to the individual's obsessions. For instance, if the individual believes that he ran over someone with his car, the individual may return to the scene over and over because he just cannot shake his doubts. The individual may even make up rules or rituals to follow that help control the anxiety he feels when having obsessive thoughts.

Typical compulsions revolve around: washing and cleaning, counting, checking, demanding reassurances, repeating actions over and over, and arranging and making items appear orderly.

OCD symptoms involving compulsions may include washing hands until the skin becomes raw, checking doors repeatedly to make sure they are locked, checking the stove repeatedly to make sure it is off, and counting in certain patterns.

What causes obsessive-compulsive disorder is not fully understood. Main theories include:

Biology. Some researchers believe OCD is a result of changes in the body's natural chemistry.

Environment. Some researchers believe that OCD stems from behavior habits that are learned over time.

Insufficient serotonin. An insufficient level of serotonin, one of the brain's chemical messengers, may contribute to obsessive compulsive disorder. Some studies that compare images of the brains of people who have obsessive-compulsive disorder with the brains of those who do not show differences in brain-activity patterns.

In addition, people with obsessive-compulsive disorder who take medications that enhance the action of serotonin often have fewer symptoms.

Strep Throat. Some studies suggest that some children develop OCD after infection with Group A beta-hemolytic streptococcal pharyngitis (strep throat). Some researchers suggest that antibody against strep throat bacteria sometimes mistakenly act as a brain enzyme. This disrupts communication between neurons in the brain and may trigger OCD. However, these studies are controversial, and more evidence is needed before strep throat can be blamed.

It was once thought that obsessive-compulsive disorder was a rare condition. But it is now known to be more common than many other mental illnesses. In fact, about 2.2 million Americans have obsessive compulsive disorder, according to the National Institute of Mental Health.

Obsessive-compulsive disorder does not affect just adults. The disorder often begins during adolescence or early childhood, usually around age 10. In adults, OCD typically begins around age 21.

OCD treatment has two main components: psychotherapy and medications.

A type of therapy called cognitive behavior therapy has been shown to be the most effective form of therapy for OCD in both children and adults. Cognitive behavior therapy involves retraining the individual thought patterns and routines so that compulsive behaviors are no longer necessary. One approach in particular is called exposure and response prevention. This therapy involves gradually exposing the individual to a feared object or obsession, such as dirt, and teaching the individual healthy ways to deal with it. Learning the techniques and new thought patterns takes effort and practice, but it is worth it. Most people with obsessive-compulsive disorder show improvement of signs and symptoms with cognitive behavior therapy.

Most people with OCD benefit from taking certain psychiatric medications. Some medications have been specifically approved by the Food and Drug Administration to treat OCD, such as the antidepressants clomipramine (ANAFRANIL®), paroxetine (PAXIL®), fluvoxamine and sertraline (ZOLOFT®).

However, many other antidepressant medications on the market may also be used to treat OCD off-label, even if they have not been specifically FDA approved for that use. Antidepressants may be helpful for OCD because they may help increase levels of serotonin, which may be deficient in OCD. All of these medications have side effects and safety concerns.

Oppositional defiant disorder (ODD) is defined as a recurrent pattern of negativistic, defiant, disobedient, and hostile behavior toward authority figures that persists for at least 6 months. Behaviors included in the definition include the following: losing one's temper; arguing with adults; actively defying requests; refusing to follow rules; deliberately annoying other people; blaming others for one's own mistakes or misbehavior; and being touchy, easily annoyed or angered, resentful, spiteful, or vindictive.

Oppositional defiant disorder often occurs along with other behavioral or mental health problems such as attention-deficit/hyperactivity disorder (ADHD), anxiety or depression. The symptoms of ODD may be difficult to distinguish from those of other behavioral or mental health problems.

There is no clear cause underpinning oppositional defiant disorder. contributing causes may include: the child's inherent temperament, the family's response to the child's style, a genetic component that when coupled with certain environmental conditions, such as lack of supervision or poor quality childcare or family instability increases the risk of ODD, a biochemical or neurological factor, the child's perception that he or she is not getting enough of the parents' time and attention.

A number of factors play a role in the development of oppositional defiant disorder. ODD is a complex problem involving a variety of influences, circumstances and genetic components. No single factor causes ODD. Possible risk factors include: having a parent with a mood or substance abuse disorder, being abused or neglected, harsh or inconsistent discipline, lack of supervision, poor relationship with one or both parents, family instability such as occurs with divorce, multiple moves, or changing schools or child care providers frequently, parents with a history of ADHD, oppositional defiant disorder or conduct problems, financial problems in the family, exposure to violence, and substance abuse in the child or adolescent.

### SUMMARY

It has been determined by the present inventor that the gastrointestinal tract of dysautonomic individuals is impaired, and that the proper levels of pancreatic enzymes and/or their precursors including the zymogens and bicarbonate ions are not present in sufficient quantities to allow proper digestion. While that impairment is relevant to the digestion of carbohydrates, fats and proteins, it is most specific and most severe with respect to protein digestion. Accordingly, while not being bound by theory, the present inventor believes that many, if not all, dysautonomias have a GI component, and thus that dysautonomias may actually have their etiology in gastrointestinal dysfunction. For example, with Guillain-Barré syndrome, it is postulated that a GI pathogen is a causative factor in the formation of the Guillain-Barré dysautonomia. Similarly, it has been found by the present inventor that populations of autistic children suffer from GI disturbances and other conditions which are dysautonomic in nature. In general, these findings represent a possible link between the etiology of autism and autonomic dysfunction. Thus, the inventor believes that other dysautonomic conditions also have GI primary etiologies.

The symptoms of dysautonomic conditions, however, may have various manifestations due to the genetic makeup of the individuals suffering from the conditions. Various gene sequences in the genetic code of the individual will result in manifestation of certain diseases or symptoms that are expressed uniquely in each individual. For example, if amino acid pool deficits due to improper protein digestion and gastrointestinal dysfunction are manifested differently in different individuals, a “disease state” may appear different depending upon the genetic makeup of the individual. Neurological expression may be all that is seen in some individuals, whereas other manifestations may demonstrate a hybrid of gastrointestinal dysfunction as well as neurological or other dysfunctions.

Accordingly, while not bound by theory, the present inventor believes that certain neurological and mental health disorders may have a dysautonomic component and that the etiology of certain neurological and mental health disorders may be related to gastrointestinal dysfunction.

Given the above, it is a goal of the present disclosure to provide therapeutic methods and pharmaceutical compositions for the treatment of the symptoms of certain neurological and mental health disorders, such as Alzheimer's disease, Bipolar disorder, OCD, and ODD. It is also a goal of the present disclosure to provide therapeutic methods and pharmaceutical compositions for the treatment of Pervasive Development Disorders such as Autism, ADD, and ADHD, and for Dysautonomias such as Familial Dysautonomia, Parkinson's, and Guillain-Barré Syndrome.

Another goal of the present disclosure is the provision of pharmaceutical compositions for the treatment of the above disorders, wherein the compositions comprise one or more digestive enzymes, e.g., one or more enzymes selected from amylases, proteases, cellulases, papaya, papain, bromelain, lipases, chymotrypsin, trypsin, and hydrolases. In some embodiments, the pharmaceutical compositions are lipid encapsulated.

Yet another goal of the present disclosure is to provide methods for making the described pharmaceutical compositions using methods such as: direct compression, microencapsulation, lipid encapsulation, wet granulation or other methods including the use of PROSOLV® (silicified microcrystalline cellulose), and other known excipients and additives to accomplish microencapsulation, lipid encapsulation, direct compression, wet or dry granulation or other suitable technology.

A further goal of the present disclosure is to provide means to deliver the pharmaceutical compositions, which can include the use of rapid dissolution (rapid dissolve), time release, or other delivery methods including oral, injection, patch, or other method. Further, the delivery of the pharmaceutical compositions may be in the form of a tablet, capsule, sprinkles, sachet, or other oral delivery method.

An additional goal of the disclosure is to demonstrate the use of fecal chymotrypsin level as a biomarker for the presence of certain neurological and mental health disorders, such as Alzheimer's disease, Bipolar disorder, OCD, and ODD, or the likelihood of an individual to develop these neurological and mental health disorders.

Accordingly, provided herein is a method for treating one or more symptoms associated with certain neurological and mental health disorders, such as Alzheimer's disease, bipolar disorder, OCD, and ODD in a patient diagnosed with any of these disorders comprising administering to the patient a therapeutically effective amount of a pharmaceutical composition comprising one or more digestive enzymes. In some embodiments, the pharmaceutical composition may be lipid-encapsulated. In some embodiments, the one or more digestive enzymes comprise one or more enzymes selected from the group consisting of proteases, amylases, cellulases, sucrases, maltases, papaya, papain, bromelain, hydrolases, and lipases. In some embodiments, the one or more digestive enzymes comprise one or more pancreatic enzymes. In some embodiments, the pharmaceutical composition comprises one or more proteases, one or more lipases, and one or more amylases. In some embodiments, the one or more proteases comprise chymotrypsin and trypsin.

The one or more digestive enzymes are, independently, derived from an animal source, a microbial source, or a plant source, or are synthetically prepared. In some embodiments, the animal source is a pig, e.g.: a pig pancreas.

In some embodiments, the pharmaceutical composition comprises at least one amylase, a mixture of proteases comprising chymotrypsin and trypsin, at least one lipase, and papain. In some embodiments, the pharmaceutical composition further comprises papaya. In some embodiments, the pharmaceutical composition comprises per dose: amylases from about 10,000 to about 60,000 U.S.P. units, proteases from about 10,000 to about 70,000 U.S.P. units, lipases from about 4,000 to about 30,000 U.S.P. units, chymotrypsin from about 2 to about 5 mg, trypsin from about 60 to about 100 mg, papain from about 3,000 to about 10,000 U.S.P. units, and papaya from about 30 to about 60 mg.

In some embodiments, the pharmaceutical composition comprises at least one protease and at least one lipase, wherein the ratio of total proteases to total lipases (in U.S.P. units) ranges from about 1:1 to about 20:1. In some embodiments, the ratio of proteases to lipases ranges from about 4:1 to about 10:1.

In some embodiments, the one or more symptoms of Alzheimer's include memory loss, language deterioration, impaired ability to mentally manipulate visual information, poor judgment, confusion, restlessness, and mood swings.

In some embodiments, the one or more symptoms of the manic phase of bipolar disorder may include euphoria, extreme optimism, inflated self-esteem, poor judgment, rapid speech, racing thoughts, aggressive behavior, agitation, increased physical activity, risky behavior, spending sprees, increased drive to perform or achieve goals, increased sexual drive, decreased need for sleep, tendency to be easily distracted, inability to concentrate, and drug abuse.

In some embodiments, the one or more symptoms of the depressive phase of bipolar disorder may include sadness, hopelessness, suicidal thoughts or behavior, anxiety, guilt, sleep problems, appetite problems, fatigue, loss of interest in daily activities, problems concentrating, irritability, chronic pain without a known cause.

In some embodiments, the one or more symptoms of OCD involving obsessions may include fear of being contaminated by shaking hands or by touching objects others have touched, doubts that the individual has locked the door or turned off the stove, repeated thoughts that the individual has hurt someone in a traffic accident, intense distress when objects are not orderly, lined up properly or facing the right way, images of hurting the individual's child, impulses to shout obscenities in inappropriate situations, avoidance of situations that can trigger obsessions, such as shaking hands, replaying pornographic images in the individual's mind, dermatitis because of frequent hand washing, skin lesions because of picking at the skin, and hair loss or bald spots because of hair pulling.

In some embodiments, the one or more symptoms of OCD involving compulsions may include washing hands until the skin becomes raw, checking doors repeatedly to make sure they are locked, checking the stove repeatedly to make sure it is off, and counting in certain patterns.

In some embodiments, the one or more behaviors included in the definition of ODD include the following: losing one's temper; arguing with adults; actively defying requests; refusing to follow rules; deliberately annoying other people; blaming others for one's own mistakes or misbehavior; and being touchy, easily annoyed or angered, resentful, spiteful, or vindictive.

In some embodiments, the pharmaceutical composition is a dosage formulation selected from the group consisting of pills, tablets, capsules, microcapsules, mini-capsules, time released capsules, mini-tabs, sprinkles, and a combination thereof.

Also provided is a method of diagnosing a patient comprising: obtaining a fecal sample from the patient, determining a level of chymotrypsin present in the fecal sample, in some cases wherein the determination is performed at 30° C., and diagnosing the patient as having a neurological or mental health disorder, such as Alzheimer's disease, Bipolar disorder, OCD, or ODD, if the determined fecal chymotrypsin level is 8.4 U/gram or less and the patient exhibits at least one symptom associated with one of these neurological or mental health disorders.

In some embodiments, the fecal chymotrypsin level is between 8.4 and 4.2 U/gram. In some embodiments, the fecal chymotrypsin level is less than 4.2 U/gram. In some embodiments, the level of chymotrypsin present in the fecal sample is determined using an enzymatic photospectrometry method. In some embodiments, the method further comprises administering to the patient an effective amount of a pharmaceutical composition comprising one or more digestive enzymes if the patient is diagnosed as having a neurological or mental health disorder, such as Alzheimer's disease, Bipolar disorder, OCD, or ODD. In some embodiments, the method further comprises determining if the administration of the pharmaceutical composition reduces one or more symptoms associated with one of these neurological or mental health disorders.

Also provided is a method of identifying a patient likely to benefit from administration of a pharmaceutical composition comprising one or more digestive enzymes comprising: obtaining a fecal sample from the patient, determining a level of chymotrypsin present in the fecal sample, in some cases wherein the determination is performed at 30° C., and identifying the patient as likely to benefit from administration of the pharmaceutical composition if the determined fecal chymotrypsin level is 8.4 U/gram or less and the patient is diagnosed with a neurological or mental health disorder, such as Alzheimer's disease, Bipolar disorder, OCD, or ODD. In some embodiments, the method further comprises determining if the patient exhibits one or more symptoms of one of these neurological or mental health disorders. In some embodiments, the benefit comprises a reduction or amelioration of one or more symptoms associated with the neurological or mental health disorder. In some embodiments, the method further comprises administering to the patient an effective amount of a pharmaceutical composition comprising one or more digestive enzymes.

Also provided is a pharmaceutical composition comprising one or more digestive enzymes, wherein the one or more digestive enzymes comprise at least one lipase and at least one protease, and wherein the ratio of total proteases to total lipases (in U.S.P. units) ranges from about 1:1 to about 20:1. In some embodiments, the ratio of total proteases to total lipases ranges from about 4:1 to about 10:1. In some embodiments, the pharmaceutical composition is lipid encapsulated.

Also provided is a pharmaceutical composition comprising at least one amylase, a mixture of proteases comprising chymotrypsin and trypsin, at least one lipase, and papain. In some embodiments, the pharmaceutical composition further comprises papaya. In some embodiments, the ratio of total proteases to total lipases ranges from about 1:1 to about 20:1.

The features and advantages described herein are not all-inclusive and, in particular, many additional features and advantages will be apparent to one of ordinary skill in the art in view of the drawings, specification, and claims. Moreover, it should be noted that the language used in the specification has been principally selected for readability and instructional purposes, and not to limit the scope of the inventive subject matter.

### DETAILED DESCRIPTION

The present disclosure provides pharmaceutical compositions and methods for treating symptoms associated with certain neurological or mental health disorders, such as Alzheimer's disease, Bipolar disorder, OCD, or ODD, Pervasive Development Disorders, and Dysautonomias. The pharmaceutical compositions described herein include one or more digestive enzymes, which are postulated by the present inventor to assist in proper digest protein and thus to ameliorate the gastrointestinal dysfunction that is associated with the described disorders.

In certain embodiments, the pharmaceutical compositions can include one or more digestive enzymes, wherein the one or more digestive enzymes comprise at least one lipase and at least one protease, and wherein the ratio of total proteases to total lipases (in U.S.P. units) ranges from about 1:1 to about 20:1. In some cases, the ratio of total proteases to total lipases ranges from about 4:1 to about 10:1. In some embodiments, the pharmaceutical composition is lipid encapsulated.

In some cases, a pharmaceutical composition for use herein comprises at least one amylase, at least one protease, and at least one lipase. In certain embodiments, the composition can comprise at least one amylase, at least two proteases, and at least one lipase. In certain embodiments the pharmaceutical composition includes multiple proteases, including, without limitation, chymotrypsin and trypsin. In certain embodiments, the composition can further include one or more hydrolases, papain, bromelain, papaya, cellulases, pancreatin, sucrases, and maltases.

The one or more enzymes can be independently derived from animal, plant, microbial, or synthetic sources. In some embodiments, the one or more enzymes are derived from pig, e.g.: pig pancreas.

One exemplary formulation for the treatment of the symptoms of neurological and mental health disorders, such as Alzheimer's disease, bipolar disorder, obsessive compulsive disorder, and oppositional defiant disorder is as follows: amylase 10,000-60,000 U.S.P. units, protease 10,000-70,000 U.S.P. units, lipase 4,000-30,000 U.S.P. units, chymotrypsin 2-5 mg, Trypsin 60-100 mg, papain 3,000-10,000 U.S.P. units/mg, and papaya 30-60 mg.

Additional formulations comprising one or more digestive enzymes may be advantageous including formulations in which the ratio of total proteases to total lipases (in U.S.P. units) is from about 1:1 to about 20:1. In some embodiments, the ratio of total proteases to total lipases is from about 4:1 to about 10:1. Such formulations are useful for treating symptoms of neurological and mental health disorders, such as Alzheimer's disease, bipolar disorder, OCD, and ODD, as well as dysautonomias (e.g., familial dysautonomia, Parkinson's, Guillain-Barré Syndrome, Aromatic-L-amino acid decarboxylase deficiency, tetrahydrobiopterin deficiency, familial paranganglioma syndrome; multiple system atrophy, dysautonomic symptoms associated with tumors such as pheochromocytoma, chemodectoma, and neuroblastoma; neurally mediated syncope, and SIDS) and pervasive development disorders such as autism, ADHD, ADD, and Asperger's.

Patients below the age of 18 are typically given a dosage such that the formulation would deliver at least 5,000 U.S.P. units of protease and no more than 10,000 U.S.P. units of lipase per kilogram weight of patient, per day. Beneficially the formulation would deliver at least 5,000 U.S.P. units of protease and no more than 7,500 U.S.P. units of lipase per kilogram weight of patient per day. Patients above the age of 18 are typically given no less than 5,000 U.S.P. units of protease per kilogram weight of patient per day.

The pharmaceutical compositions can be formulated in dosage forms for any route of administration, including oral, parenteral, IV, inhalation, and buccal dosage formulations. In certain embodiments, a dosage formulation may be administered by an oral preparation including, but not limited to, an encapsulated tablet, mini-tabs, microcapsule, mini-capsule, time released capsule, sprinkle or other methodology. In one embodiment, the oral preparation is encapsulated using one or more lipids. Alternatively, the oral preparation may be encapsulated using enteric coating or organic polymers. A formulation may also be prepared using PROSOLV® technology, direct compression, dry granulation, wet granulation, and/or a combination of these methods.

Fecal chymotrypsin level is a sensitive, specific measure of proteolytic activity, see e.g.: U.S. Pat. No. 6,660,831, incorporated by reference herein. Normal levels of chymotrypsin are considered to be greater than 8.4 U/gram. Decreased values (less than 4.2 U/gram) suggest diminished pancreatic output (pancreatic insufficiency), hypoacidity of the stomach or cystic fibrosis. Elevated chymotrypsin values suggest rapid transit time, or less likely, a large output of chymotrypsin from the pancreas.

For the fecal chymotrypsin test, a stool sample is collected from each of the subjects. Each stool sample can be analyzed using an enzymatic photospectrometry analysis to determine the level of fecal chymotrypsin in the stool; in some cases, the assay is performed at 30° C., see, e.g.: U.S. Pat. No. 6,660,831, incorporated by reference herein. Alternatively, other methods, such as the colorimetric method, use of substrates, use of assays, and/or any other suitable method may be used to measure the fecal chymotrypsin levels. The levels of fecal chymotrypsin in the samples of the individuals suspected of or diagnosed as having neurological and mental health disorders, such as Alzheimer's disease, Bipolar disorder, OCD, and ODD, are compared to the levels of fecal chymotrypsin in individuals not suspected or diagnosed with these neurological and mental health disorders to determine if the tested individuals exhibit lower fecal chymotrypsin values and to determine if the individuals would benefit from the administration of a composition as described herein.

The foregoing description of the embodiments of the invention has been presented for the purposes of illustration and description. It is not intended to be exhaustive or to limit the invention to the precise form disclosed. Many modifications and variations are possible in light of this disclosure. It is intended that the scope of the invention be limited not by this detailed description, but rather by the claims appended hereto.

What is claimed is:

1. A method for treating one or more symptoms associated with a neurological or mental health disorder in a patient diagnosed with the neurological or mental health disorder comprising administering to the patient a therapeutically effective amount of a pharmaceutical composition comprising one or more digestive enzymes.

2. The method of claim 1, wherein the neurological or mental health disorder is selected from the group consisting of Alzheimer's disease, bipolar disorder, obsessive compulsive disorder, oppositional defiant disorder, and a combination thereof.

3. The method of claim 1, wherein the one or more digestive enzymes comprise one or more enzymes selected from the group consisting of proteases, amylases, cellulases, sucrases, maltases, papaya, papain, and lipases.

4. The method of claim 1, wherein the one or more digestive enzymes comprise one or more pancreatic enzymes.

5. The method of claim 3, wherein the proteases comprise chymotrypsin and trypsin.

6. The method of claim 1, wherein the one or more digestive enzymes are, independently, derived from an animal source, a microbial source, or a plant source, or are synthetically prepared.

7. The method of claim 6, wherein the animal source is a pig.

8. The method of claim 1, wherein the pharmaceutical composition comprises at least one amylase, a mixture of proteases comprising chymotrypsin and trypsin, at least one lipase, and papain.

9. The method of claim 8, wherein the pharmaceutical composition further comprises papaya.

10. The method of claim 1, wherein the pharmaceutical composition comprises: amylases from about 10,000 to about 60,000 U.S.P, proteases from about 10,000 to about 70,000 U.S.P, lipases from about 4,000 to about 30,000 U.S.P, chymotrypsin from about 2 to about 5 mg, trypsin from about 60 to about 100 mg, papain from about 3,000 to about 10,000 U.S.P. units, and papaya from about 30 to about 60 mg.

11. The method of claim 1, wherein the pharmaceutical composition comprises at least one protease and at least one lipase, and wherein the ratio of total proteases to total lipases (in U.S.P. units) ranges from about 1:1 to about 20:1.

12. The method of claim 11, wherein the ratio of proteases to lipases ranges from about 4:1 to about 10:1.

13. The method of claim 2, wherein the one or more symptoms of Alzheimer's disease is selected from the group consisting of memory loss, language deterioration, impaired ability to mentally manipulate visual information, poor judgment, confusion, restlessness, mood swings, and a combination thereof.

14. The method of claim 2, wherein the one or more symptoms of the manic phase of bipolar disorder is selected from the group consisting of euphoria, extreme optimism, inflated self-esteem, poor judgment, rapid speech, racing thoughts, aggressive behavior, agitation, increased physical activity, risky behavior, spending sprees, increased drive to perform or achieve goals, increased sexual drive, decreased need for sleep, tendency to be easily distracted, inability to concentrate, drug abuse, and a combination thereof.

15. The method of claim 2, wherein the one or more symptoms of the depressive phase of bipolar disorder is selected from the group consisting of sadness, hopelessness, suicidal thoughts or behavior, anxiety, guilt, sleep problems, appetite problems, fatigue, loss of interest in daily activities, problems concentrating, irritability, chronic pain without a known cause, and a combination thereof.

16. The method of claim 2, wherein the one or more symptoms of OCD involving obsessions is selected from the group consisting of fear of being contaminated by shaking hands or by touching objects others have touched, doubts that the individual has locked the door or turned off the stove, repeated thoughts that the individual has hurt someone in a traffic accident, intense distress when objects are not orderly, lined up properly or facing the right way, images of hurting the individual's child, impulses to shout obscenities in inappropriate situations, avoidance of situations that can trigger obsessions, such as shaking hands, replaying pornographic images in the individual's mind, dermatitis because of frequent hand washing, skin lesions because of picking at the skin, hair loss or bald spots because of hair pulling, and a combination thereof.

17. The method of claim 2, wherein the one or more symptoms of OCD involving compulsions is selected from the group consisting of washing hands until the skin becomes raw, checking doors repeatedly to make sure they are locked, checking the stove repeatedly to make sure it is off, counting in certain patterns, and a combination thereof.

18. The method of claim 2, wherein the one or more symptoms included in the definition of ODD is selected from the group consisting of losing one's temper; arguing with adults; actively defying requests; refusing to follow rules; deliberately annoying other people; blaming others for one's own mistakes or misbehavior; being touchy, easily annoyed or angered, resentful, spiteful, or vindictive, and a combination thereof.

19. The method of claim 1, wherein the pharmaceutical composition is a dosage formulation selected from the group consisting of pills, tablets, capsules, microcapsules, mini-capsules, time released capsules, mini-tabs, sprinkles, and a combination thereof.

20. A method of diagnosing a patient comprising:
obtaining a fecal sample from the patient;
determining a level of chymotrypsin present in the fecal sample; and
diagnosing the patient as having a neurological or mental health disorder if the determined fecal chymotrypsin level is 8.4 U/gram or less and the patient exhibits at least one symptom associated with the neurological or mental health disorder,
wherein the neurological or mental health disorder is selected from the group consisting of Alzheimer's disease, bipolar disorder, obsessive compulsive disorder, oppositional defiant disorder, and a combination thereof.

21. The method of claim 20, wherein the fecal chymotrypsin level is between about 8.4 and about 4.2 U/gram.

22. The method of claim 20, wherein the fecal chymotrypsin level is less than 4.2 U/gram.

23. The method of claim 20, wherein the level of chymotrypsin present in the fecal sample is determined using an enzymatic photo spectrometry method.

24. The method of claim 20, further comprising administering to the patient an effective amount of a pharmaceutical composition comprising one or more digestive enzymes if the patient is diagnosed as having the neurological or mental health disorder.

25. The method of claim 24, further comprising determining if the administration of the pharmaceutical composition reduces or ameliorates one or more symptoms associated with the neurological or mental health disorder.

26. The method of claim 25, further comprising comparing the post-administration measurement of one or more symptoms of the neurological or mental health disorder to a pre-administration measurement of the one or more symptoms of the neurological or mental health disorder.

27. A method of identifying a patient likely to benefit from administration of a pharmaceutical composition comprising one or more digestive enzymes comprising:
obtaining a fecal sample from the patient;
determining a level of chymotrypsin present in the fecal sample; and
identifying the patient as likely to benefit from administration of the pharmaceutical composition if the determined fecal chymotrypsin level is 8.4 U/gram or less and the patient is diagnosed with a neurological or mental health disorder,
wherein the neurological or mental health disorder is selected from the group consisting of Alzheimer's disease, bipolar disorder, obsessive compulsive disorder, oppositional defiant disorder, and a combination thereof.

28. The method of claim 27, further comprising determining if the patient exhibits one or more symptoms of the neurological or mental health disorder.

29. The method of claim 27, wherein the benefit comprises a reduction or amelioration of one or more symptoms associated with the neurological or mental health disorder.

30. The method of claim 27, wherein the level of chymotrypsin present in the fecal sample is determined using an enzymatic photo spectrometry method.

31. The method of claim 27, further comprising administering to the patient an effective amount of a pharmaceutical composition comprising one or more digestive enzymes.

32. A pharmaceutical composition comprising one or more digestive enzymes, wherein the one or more digestive enzymes comprise at least one lipase and at least one protease, and wherein the ratio of total proteases to total lipases (in U.S.P. units) ranges from about 1:1 to about 20:1.

33. The pharmaceutical composition of claim 32, wherein the ratio of total proteases to total lipases ranges from about 4:1 to about 10:1.

34. A pharmaceutical composition comprising at least one amylase, a mixture of proteases comprising chymotrypsin and trypsin, at least one lipase, and papain.

35. The pharmaceutical composition of claim 34, wherein the pharmaceutical composition further comprises papaya.

36. The pharmaceutical composition of claim 34, wherein the ratio of total proteases to total lipases ranges from about 1:1 to about 20:1.

37. A pharmaceutical preparation for treating an individual exhibiting one or more symptoms of a neurological or mental health disorder comprising a therapeutically effective amount of a digestive enzyme, wherein the neurological or mental health disorder is selected from the group consisting of Alzheimer's disease, bipolar disorder, obsessive compulsive disorder, oppositional defiant disorder, and a combination thereof.

38. The pharmaceutical preparation of claim 37, wherein the digestive enzyme is selected from the group consisting of amylase, lipase, protease, and a combination thereof.

39. The pharmaceutical preparation of claim 37, wherein the digestive enzyme is further selected from the group consisting of chymotrypsin, trypsin, pancreatin, papaya, papain, and a combination thereof.

40. The pharmaceutical preparation of claim 37, wherein the enzyme is derived from a source selected from the group consisting of animal enzymes, plant enzymes, synthetic enzymes, and a combination thereof.

41. The pharmaceutical preparation of claim 37, wherein the preparation is manufactured using a technology selected from the group consisting of PROSOLV® technology, enteric coating, lipid encapsulation, direct compression, dry granulation, wet granulation, and a combination thereof.

42. The pharmaceutical preparation of claim 37, wherein the preparation is administered orally via a dosage formulation selected from the group consisting of pills, tablets, capsules, microcapsules, mini-capsules, time released capsules, mini-tabs, sprinkles, and a combination thereof.

43. The pharmaceutical preparation of claim 38, wherein the amount of amylase ranges from about 10,000 to about 60,000 U.S.P. units/mg.

44. The pharmaceutical preparation of claim 38, wherein the amount of protease ranges from about 10,000 to about 70,000 U.S.P. units/mg.

45. The pharmaceutical preparation of claim 38, wherein the amount of lipase ranges from about 4,000 to about 30,000 U.S.P. units/mg.

46. The pharmaceutical preparation of claim 39, wherein the amount of pancreatin ranges from about 2,000 to about 6,000 U.S.P. units/mg.

47. The pharmaceutical preparation of claim 39, wherein the amount of chymotrypsin ranges from 2 to 5 mg.

48. The pharmaceutical preparation of claim 39, wherein the amount of papain ranges from about 3,000 to about 10,000 U.S.P. units/mg.

49. The pharmaceutical preparation of claim 39, wherein the amount of papaya ranges from about 30 to about 60 mg.

50. The pharmaceutical preparation of claim 39, wherein the amount of trypsin ranges from about 60 to about 100 mg.

51. The pharmaceutical preparation of claim 37, wherein a symptom of the neurological or mental health disorder is ameliorated.

52. The method of claim 51, wherein the symptom of Alzheimer's disease is selected from the group consisting of memory loss, language deterioration, impaired ability to mentally manipulate visual information, poor judgment, confusion, restlessness, mood swings, and a combination thereof.

53. The method of claim 51, wherein the symptom of the manic phase of bipolar disorder is selected from the group consisting of euphoria, extreme optimism, inflated self-esteem, poor judgment, rapid speech, racing thoughts, aggressive behavior, agitation, increased physical activity, risky behavior, spending sprees, increased drive to perform or achieve goals, increased sexual drive, decreased need for sleep, tendency to be easily distracted, inability to concentrate, drug abuse, and a combination thereof.

54. The method of claim 51, wherein the symptom of the depressive phase of bipolar disorder is selected from the group consisting of sadness, hopelessness, suicidal thoughts or behavior, anxiety, guilt, sleep problems, appetite problems, fatigue, loss of interest in daily activities, problems concentrating, irritability, chronic pain without a known cause, and a combination thereof.

55. The method of claim 51, wherein the symptom of OCD involving obsessions is selected from the group consisting of fear of being contaminated by shaking hands or by touching objects others have touched, doubts that the individual has locked the door or turned off the stove, repeated thoughts that the individual has hurt someone in a traffic accident, intense distress when objects are not orderly, lined up properly or facing the right way, images of hurting the individual's child, impulses to shout obscenities in inappropriate situations, avoidance of situations that can trigger obsessions, such as shaking hands, replaying pornographic images in the individual's mind, dermatitis because of frequent hand washing, skin lesions because of picking at the skin, hair loss or bald spots because of hair pulling, and a combination thereof.

56. The method of claim 51, wherein the symptom of OCD involving compulsions is selected from the group consisting of washing hands until the skin becomes raw, checking doors repeatedly to make sure they are locked, checking the stove repeatedly to make sure it is off, counting in certain patterns, and a combination thereof.

57. The method of claim 51, wherein the symptom included in the definition of ODD is selected from the group consisting of losing one's temper; arguing with adults; actively defying requests; refusing to follow rules; deliberately annoying other people; blaming others for one's own mistakes or misbehavior; being touchy, easily annoyed or angered, resentful, spiteful, or vindictive, and a combination thereof.

58. A method of treating an individual having a neurological or mental health disorder with a therapeutically effective amount of digestive enzymes comprising the steps of:
measuring a level of fecal chymotrypsin in a stool sample of the individual;
comparing the level of fecal chymotrypsin with a normal fecal chymotrypsin level; and
administering the digestive enzymes to the individual if the level of fecal chymotrypsin in the individual is less than the normal fecal chymotrypsin level
wherein the neurological or mental health disorder is selected from the group consisting of Alzheimer's disease, bipolar disorder, obsessive compulsive disorder, oppositional defiant disorder, and a combination thereof.

59. The method of claim 58, further comprising the steps of:
administering the digestive enzymes to the individual in order to promote protein digestion; and
administering the digestive enzymes to the individual in order to ameliorate a symptom of the neurological or mental health disorder.

60. The method of claim 58, wherein the stool sample is measured using a technique selected from the group consisting of enzymatic photospectrometry, colorimetry, treatment with substrates, assays, and a combination thereof.
